(19) United States
(12) Patent Application Publication
(10) Pub. No.: US 2007/0106245 A1
(43) Pub. Date: May 10, 2007

(54) Infusion guidewire

(75) Inventors: Colleen McQueen, Fremont, CA (US); Arlene Errazo, Sunnyvale, CA (US); Hung Ha, San Jose, CA (US); Casidy Hallsten, San Mateo, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Kerberos Proximal Solutions, Inc., Cupertino, CA (US)

(21) Appl. No.: 11/270,851

(22) Filed: Nov. 8, 2005

(51) Int. Cl. A61M 31/00

(52) U.S. Cl. 604/508; 604/523

(57) ABSTRACT

An infusion guidewires assemblies and methods are provided that can be used for infusion of fluid at desired location within body lumen of a patient and to facilitate placement of catheters and other devices into a body lumen of a patient. The infusion guidewire assembly includes a guidewire shaft having a distal end, a proximal end and a fluid delivery lumen therethrough. The guidewire assembly further includes an infusion hub adapted to be removably mounted on the proximal end of the guidewire shaft, wherein the hub provides fluid connection to the fluid delivery lumen; and a guidewire coil attached to the distal end of the guidewire shaft. The guidewire shaft of the assembly includes a plurality of infusion ports formed over a distal region proximal to the guidewire coil.

### BACKGROUND OF THE INVENTION

The present invention relates generally to catheters and guidewires of the type used in the medical profession for infusion of fluids and/or medicaments to a tissue site within the body and, more specifically, the present invention provides an improved infusion guidewire that can be used both in the infusion of a fluid at desired location within body lumen of a patient and can function to facilitate placement of catheters and other devices into a body lumen of a patient.

Medical guidewires and catheters are devices that can be introduced into and navigated through blood vessels and other narrow passages in the body. These devices can be navigated through the blood vessels until the distal end of the device is positioned in the desired location. Guidewires are typically used for introduction of a catheter over the guidewire.

One of the purposes for a guidewire is the positioning of a catheter used to infuse a liquid at a given location in a patient's blood vessel (i.e., infusion catheter). Cardiovascular guidewires used for this purpose are typically constructed to have a solid core wire and are dimensioned such that they are capable of being received within a catheter lumen as the catheter is advanced over the guidewire. Thus, a guidewire is typically first inserted into a blood vessel and an operator (e.g., physician) controls the advancement and resulting position of the distal end of the guidewire by manipulations performed at the proximal end outside the body. Then, when the guidewire is positioned in the desired location, a catheter is advanced over the guidewire, and the guidewire may either be left in place or withdrawn to leave only the catheter in the blood vessel. One distinct advantage of guidewires in general, is their ability to go into small lumens at distal locations. They allow access to remote areas with the ability to guide larger devices into place. Additionally, they can also pass through very narrow openings, such as severely stenosed vessels.

Infusion catheters generally include a hollow lumen that functions both to receive a guidewire and to allow passage of fluid therefrom and a fluid outlet for delivery of fluid at a distal portion of the catheter. An exemplary perfusion catheter is described in U.S. Pat. No. 6,669,662, wherein the catheter includes a shaft having a lumen and a plurality of ports through the shaft. The catheter further includes a rotatable core with a core groove disposed within the lumen. The plurality of ports can be exposed by retraction of the core and the core can be rotated within the shaft. U.S. Pat. No. 6,063,069 describes an infusion catheter used for power lysis of thrombus. The infusion catheter is formed with a catheter lumen extending from a proximal end connector assembly to a distal end valve that is normally closed but is penetrable to allow introduction of a guidewire through the catheter lumen and a distal end valve to allow advancement of the infusion catheter over the guidewire, thereby allowing access of the distal infusion segment to the desired location.

Infusion catheters, however, typically do not possess the requisite rigidity necessary for unaided advancement in a blood vessel and, therefore, must be used in conjunction with a guidewire. Because the multiple steps of using an infusion catheter in conjunction with a guidewire have disadvantages (e.g., increased procedure time, increased invasiveness, etc.), attempts have been made to fabricate a device that can be advanced in a patient's blood vessel without the use of an additional guidewire and which also functions for infusing a liquid. As a result, infusion devices have been produced having the rigidity required for advancement through a patient's blood vessel and include a hollow lumen for carrying and infusing a liquid. Such devices often are a hybrid of multiple components that are found in standard guidewires and catheters. In particular, the devices typically include a wire coil portion and a sheath portion either surrounding the wire coil or positioned within the wire coil, with the two portions defining a lumen therein. Illustrative examples are provided as follows:

U.S. Pat. No. 5,211,636 teaches an infusion guidewire with a guidewire body having a helical coil with a flexible nonporous sheath within the lumen. The helical coil is close wound along the majority of its length, but the distal end of the helical coil has spaced windings to permit infusion of the fluid.

U.S. Pat. No. 6,027,461 discloses an infusion guidewire/catheter having an integral, tapered core wire within an infusion lumen formed of the aligned lumens of the conduit of a proximal connector housing, a proximal inner sheath in a proximal guidewire portion of a distal coil wire in a distal guidewire portion. A plurality of infusion side holes are formed in a distal infusion segment of the outer sheath in fluid communication with the infusion lumen. A similar device is taught in U.S. Pat. No. 5,997,487.

U.S. Pat. No. 4,932,419 teaches a flexible catheter-like guidewire which has a multi-filar, cross-wound coil body terminating in a distal ball tip element. The body has an inner coil and an outer coil, each formed by multiple coil wires, that define an elongated lumen, which can hold a fluid for deliver to the distal tip of the device.

U.S. Pat. No. 6,059,767 teaches medical guidewires and catheters that include tubular bodies having coils at their distal ends to permit steering through the vasculature, where the devices are used for delivering fluids to a target site, draining fluids, and infusing medicaments.

Despite these advances, a need remains for improved infusion guidewire assemblies and methods that allow rapid, controlled, and efficacious infusion of a liquid to a desired location in a body lumen or vessel. For example, the multiple sheath and coil layer construction of the existing devices can restrict the cross-sectional lumen area available for fluid deliver. Furthermore, coiled layers making up large portions the guidewire shafts, including the infusion segments, can limit controlled infusion due to bending, contraction and expansion of the coils. Therefore, there is a need for improved infusion assemblies that are dimensioned such that they are suitable for use as a traditional guidewire (e.g., introduction and positioning of other devices, such as a catheter) as well as for infusion of a liquid at a desired location in a body lumen, and wherein the infusion assembly does not require a guidewire shaft having multiple sheath and coil layers.

### BRIEF SUMMARY OF THE INVENTION

In one aspect of the invention, an infusion guidewire assembly is provided. The infusion guidewire assembly includes a guidewire shaft having a distal end, a proximal end and a fluid delivery lumen therethrough. The guidewire assembly further includes an infusion hub adapted to be removably mounted on the proximal end of the guidewire shaft, wherein the hub provides fluid connection to the fluid delivery lumen; and a guidewire coil attached to the distal end of the guidewire shaft. The guidewire coil may be shapeable or may have a preformed deflection, and in either case will usually be isolated from the fluid delivery lumen of the guidewire shaft. The guidewire shaft of the assembly includes a plurality of infusion ports formed over a distal region proximal to the guidewire coil.

The guidewire shaft, according to the present invention, is dimensioned as to maintain the general functionality of a guidewire as understood in the medical arts. In particular, the guidewire shaft of the assembly can be advanced or positioned in a body lumen of a patient (e.g., blood vessel), for example, by application of manual compression, tension and torque to the proximal end of the guidewire which remains outside the patient. Furthermore, once the guidewire is positioned in the desired location within a body lumen, a catheter or other device can optionally be advanced over the guidewire shaft.

In one embodiment, for example, the guidewire shaft has an outer diameter in the range of about 0.25 mm to about 1.25 mm. The guidewire shaft further includes a fluid delivery lumen therethrough which is dimensioned to fluidically connect the proximal end of the guidewire shaft (typically via the hub) with the infusion ports. The size of the fluid delivery lumen of a shaft can vary, for example, according to the desired application of the guidewire assembly, as well as the properties of a fluid (e.g., viscosity) to be disposed in the delivery lumen in a particular application of the assembly. In one embodiment, for example, the guidewire shaft includes a fluid delivery lumen with a diameter in the range of about 0.15 mm to about 1 mm.

The fluid infusion ports are formed over a distal region of the guidewire shaft and will be dimensioned such that a fluid disposed in the fluid delivery lumen can be infused from the fluid delivery lumen through the ports and infused into an area outside the guidewire shaft. The size of the infusion ports can vary and the port size selected may depend, for example, on the intended application of the guidewire assembly and/or the properties of a fluid (e.g., viscosity) to be disposed in the guidewire of the invention and infused through the ports. In one embodiment, for example, the infusion ports can have a width in the range from about 0.05 mm to about 0.4 mm. The geometry of the infusion ports can vary. Often, the ports will have a generally circular shape so that the width is equal to the diameter. In other cases, however, they may have an ovoid or other regular non-circular geometry. In other instances, the ports could be asymmetric and/or have irregular geometries. The range of widths set forth above will generally refer to the maximum width across the center or centroid of the infusion port.

The plurality of infusion ports are formed over a distal region of the guidewire shaft that is proximate to the guidewire coil. Both the orientation of the ports and the pattern of distribution over a region of the guidewire shaft can vary, for example, based on the intended use of the assembly. The infusion ports, for example, can be in the form of slots, holes, or any geometrical configuration suitable for infusing a liquid from the lumen of the guidewire shaft to an area adjacent to the ports. The infusion ports can be densely formed over a relatively short segment (e.g., 0.2 cm to 5 cm) of the distal region or can be more sparsely populated over a longer segment including, for example, up to the entire length of the guidewire shaft. In one embodiment, the plurality of ports can include 1 to 50 infusion ports spaced over a distal length in the range from about 0.5 cm to about 15 cm. Ports can be oriented and distributed circumferentially and/or in a longitudinal fashion on the shaft of the wire or oriented in a spiral configuration on the guidewire shaft.

In one embodiment of the invention, the infusion guidewire assembly can further include an injection device which may be removably connected to the infusion hub. The infusion hub, according to the current invention, typically includes a distal end that can be removably connected to and in fluid communication with the guidewire shaft, for example, at the proximal end of the guidewire shaft. The proximal end of the infusion hub can be removably connected to an injection device. The infusion hub can further include a fluid input port located between the proximal and distal ends of the infusion hub. In operation, fluid may be introduced into the infusion hub via the fluid input port or the injection device through the proximal end of the infusion hub. The injection device may include, for example, a fitting or fluid port for connection of a reservoir or fluid source to the infusion guidewire assembly. In one embodiment, the injection device includes a syringe. In other embodiments, the injection device may include an indaflator, a pump, an infusion pouch, or the like.

In another aspect, the invention includes a method treating a body lumen, typically a blood vessel. The method includes providing an infusion guidewire assembly, positioning a shaft of the assembly in a body lumen, introducing an interventional catheter over the guidewire while the guidewire remains positioned within the body lumen, and infusing an agent through infusion ports in a shaft of the guidewire proximal to a distal guidewire coil.

A fluid introduced into a body lumen of a patient can comprises a variety of agents, including, for example, pharmaceutically active agents or medicaments suitable for treating a body lumen or ameliorating a condition in the body lumen. In one embodiment, the agent is a thrombolyitc agent. In other embodiments, the fluid can include agents that inhibit, e.g. prevent or ameliorate or conditions such restenosis.

### BRIEF DESCRIPTION OF THE DRAWINGS

FIG. 1 is a cross-sectional view of a guidewire infusion assembly according to one embodiment of the present invention.

FIGS. 2A through 2C illustrate different arrangements of the infusion ports over the distal end of the guidewire shaft.

FIG. 3 illustrates use of the infusion guidewire for delivering an agent into a blood vessel in accordance with the methods of the present invention.

### DETAILED DESCRIPTION OF THE INVENTION

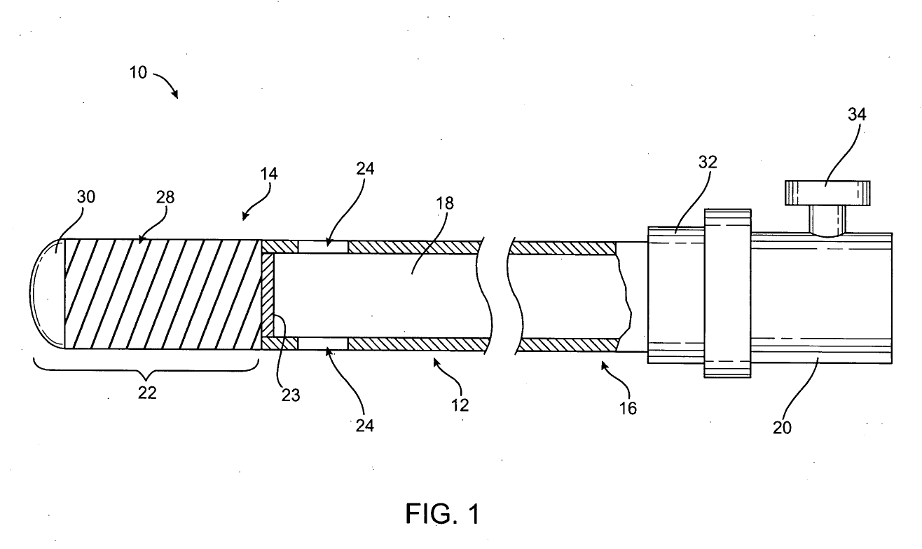

FIG. 1 is a cross-sectional view of a guidewire infusion assembly 10 according to an embodiment of the present invention. The assembly includes a guidewire shaft 12 having a distal end 14, a proximal end 16, and a fluid delivery lumen 18. An infusion hub 20 can be adapted to be removably mounted on the proximal end 16 of the guidewire shaft 12. The infusion hub 20 provides a fluid connection to the fluid delivery lumen 18. A guidewire coil 22 additionally is attached to the distal end 14 of the guidewire shaft 12. The shaft 12 of the guidewire includes a plurality of ports 24 formed over a distal region of the shaft 12 that is proximal to the guidewire coil 22. Usually, but not necessarily, the fluid delivery lumen 18 will be isolated from the guidewire coil 22, for example by a barrier 23 at the distal end of the guidewire shaft 12. In this way, all fluid delivered through the fluid delivery lumen 18 will be infused into the target body lumen via the plurality of ports 24.

The guidewire shaft 12 is of a composition and design such that the guidewire shaft 12 of the assembly 10 can be advanced or positioned in a body lumen of a patient, for example, by application of manual compression, tension and/or torque to the proximal end of the guidewire, e.g. by manually manipulating the hub 20. The guidewire shaft 12 can be composed of any material having flexibility and torqueability suitable for use of the guidewire as described herein, while maintaining a small cross sectional diameter (e.g., less than about 1.5 mm). In one embodiment, the guidewire shaft 12 is composed of a nickel titanium alloy material marketed under the trademark “NITINOL”. In other embodiments, the guidewire shaft 12 may be composed of a spring stainless steel.

The shaft 12 of the guidewire is dimensioned as to permit use of the guidewire assembly 10 for advancement and positioning, as described above, and for delivery of a fluid through the fluid delivery lumen 18. In one embodiment, the guidewire shaft 12 has an outer diameter in the range from about 0.25 mm to about 1.25 mm. The diameter of the fluid deliver lumen 18 of the guidewire shaft 12 can vary and may be selected, for example based on the intended application of the device and/or the properties of the fluid utilized (e.g., viscosity). In one embodiment, the fluid delivery lumen 18 is in the range of about 0.15 mm to about 1 mm. The length of the guidewire will depend on its intended use. For vascular uses, the length may vary in the range from 50 cm to 300 cm, typically being in the range from 60 cm to 190 cm.

In one embodiment, the distal end of the assembly includes a guidewire coil 22, which can aid the positioning of the guidewire through a body lumen of a patient. The guidewire coil 22 is constructed of a flexible coil segment 28, wherein the distal end of the coil segment 28 terminates in a tip 30. The tip 30 can be rounded in order to allow the tip 30 to more easily slide past a contacted surface, such as an interior wall of a patient's body lumen (e.g., blood vessel wall). The coil segment 28 is flexible and capable of bending laterally. In some instances, the coil segment 28 may be shapeable so that a user may pre-shape the tip into the desired non-linear configuration. In other instances, the coil segment 28 may have a permanent preformed bend or deformation to prevent steering of the guidewire through the vasculature or other body lumens.

The guidewire assembly 10 further includes an infusion hub 20 adapted to be removably mounted on the proximal end 16 of the guidewire shaft 12. The infusion hub 20 provides fluid connection to the fluid delivery lumen 18 and can optionally be removed, for example, to allow the infusion guidewire to function as an ordinary guidewire to facilitate placement of other devices (e.g., catheters), though removal is not necessarily required for such use. In one embodiment, the infusion hub 20 is mounted to the proximal end 16 of the guidewire shaft 12 via a valved connection 32, such as a hemostasis valve. The infusion hub 20 according to the invention typically includes at least one fluid port 34 suitable for the input and/or output of fluid. The fluid port 34 can further be fluidically connected with a fluid reservoir, syringe, or other fluid source.

As noted above, the guidewire shaft 12 includes a plurality of fluid infusion ports 24 formed over a distal region of the shaft 12 that is proximal to the guidewire coil 22. The size, number, distribution, and shape of the plurality of infusion ports 24 is varied in alternate embodiments depending upon the intended application of the guidewire assembly, including, for example, the flow rate to be achieved and/or the properties of the fluid (e.g., viscosity) to be infused through he ports 24. In one embodiment, the infusion ports 24 can have a width in the range of about 0.05 mm to about 0.4 mm.

The infusion ports 24 are of any geometrical configuration suitable for fluid flow through the ports 24, including infusion of a liquid from the lumen 18 to an area adjacent to the ports 24 and can include, for example, slots, holes, etc. The infusion ports 24 can be formed over guidewire distal region segments of varying length, including, for example, a relatively short segment (e.g., about 0.2 cm to about 5 cm) to relatively longer segments (e.g., greater than 5 cm and up to the entire length of the guidewire shaft). The density of infusion port distribution over the guidewire shaft can vary according to the desired application of the assembly. In one embodiment, the plurality of ports can include 1 to about 50 infusion ports spaced over a distal length in the range from about 0.5 cm to about 15 cm. Ports can be oriented and distributed circumferentially and/or in a longitudinal fashion on the shaft of the wire or arranged in a spiral or helical configuration on the guidewire shaft.

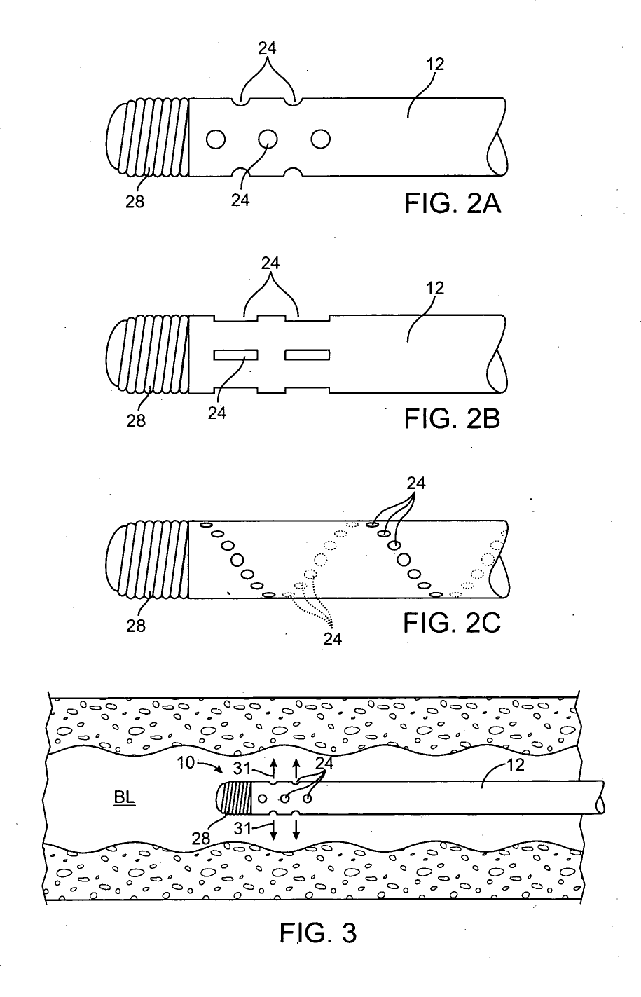

Referring now to FIGS. 2A through 2C, exemplary circular perfusion ports 24 on guidewire shaft 12 are illustrated in FIG. 2A. The circular ports 24 are arranged in axial lines spaced apart by 90° on four quadrants of the shaft. While only two or three ports 24 are shown on each axial line, it will be appreciated that greater or lesser numbers could also be employed. The guidewire shaft 12 having rectangularly slotted infusion ports 24 is illustrated in FIG. 2B. Again, the rectangular slotted ports 24 are arranged in four axial lines on 90° quadrants of the shaft. The number of ports may vary and the spacing may also vary. Referring now to FIG. 2C, smaller circular ports 24 arranged in a spiral pattern over the distal region of the shaft 12 are illustrated.

In use, the distal end of the guidewire is introduced into a body lumen BL (FIG. 3) of a patient (e.g., blood vessel) and the shaft 12 is positioned in the desired location. Once the shaft 12 is in position, a fluid is introduced into the fluid delivery lumen 18 and directed out of the fluid infusion ports 24, as shown by arrows 31, thereby delivering the fluid to an area of the body lumen proximate to the infusion ports 24. Alternatively, it will be understood that the assembly might be utilized in removing fluid from a body lumen rather than introducing a fluid into a body lumen. In such an embodiment, fluid in the body lumen is drawn through the infusion ports 24 and into the fluid lumen 18, for example, by application of negative pressure to the fluid lumen.

Fluid infusion may be achieved by a variety of conventional techniques. For example, fluid may be infused by connecting port 34 to a fluid-filled bag which is suspended sufficiently high above the patient so that the fluid will flow under gravity. Alternatively, port 34 may be connected to a peristaltic or other fluid pump which produces a positive pressure which will cause the fluid to flow through the lumen 18 and out of the infusion ports 24. As a third alternative, the port 34 may be connected to a syringe for manual introduction of the fluid.

A fluid infused according to the present invention can include a variety of agents and is not intended to be limed to any particular agent or class of agents. An infused fluid can include pharmacologically active agents or medicaments suitable for treating, ameliorating, or preventing a variety of conditions, such as stenosis. An agent can include a thromolytic agent, such as plasminogen activators or heparin compounds. Alternatively, a fluid can be saline or other biologically neutral fluid and/or may be used primarily for rinsing a body lumen. Agents can include imaging agents or contrast agents used for medical imaging purposes.

It is understood that the examples and embodiments described herein are for illustrative purposes only and that various modifications or changes in light thereof will be suggested to persons skilled in the art and are to be included within the spirit and purview of this application and scope of the appended claims. Numerous different combinations are possible, and such combinations are considered to be part of the present invention.

What is claimed is:

1. An infusion guidewire assembly comprising:
a guidewire shaft having a distal end, a proximal end, and a fluid delivery lumen therethrough;
an infusion hub adapted to be removably mounted on the proximal end of the guidewire shaft, wherein said hub provides fluid connection to the fluid delivery lumen; and
a guidewire coil attached to the distal end of the guidewire shaft;
wherein the guidewire shaft has a plurality of infusion ports formed over a distal region proximal to the guidewire coil.

2. An infusion guidewire assembly as in claim 1, wherein the guidewire shaft has an outer diameter in the range from 0.25 mm to 1.25 mm and a fluid delivery lumen in the range 0.15 mm to 1.00 mm.

3. An infusion guidewire assembly as in claim 2, wherein the infusion ports have a width in the range from 0.05 mm to 0.4 mm.

4. An infusion guidewire assembly as in claim 2, wherein the ports are oriented circumferentially and in a longitudinal fashion on the guidewire shaft.

5. An infusion guidewire assembly as in claim 2, wherein the ports are oriented in a spiral configuration on the guidewire shaft.

6. An infusion guidewire assembly as in claim 1, wherein the infusion ports are a plurality of holes.

7. An infusion guidewire assembly as in claim 1, wherein from 1 ports to 50 ports are spaced over a distal length in the range from 0.5 cm to 15 cm.

8. An infusion guidewire assembly as in claim 1, further comprising a injection device which may be removable connected to the infusion hub.

9. An infusion guidewire assembly as in claim 8, wherein the injection device is a syringe.

10. An infusion guidewire assembly as in claim 8, wherein the injection device is an indeflator.

11. An infusion guidewire assembly as in claim 8, wherein the injection device comprises a fitting for connection of a fluid source.

12. A method for treating a body lumen, said method comprising:
providing an infusion guidewire assembly;
positioning a shaft of the assembly in a body lumen;
introducing an interventional catheter over the guidewire while said guidewire remains positioned within said body lumen; and
infusing an agent through a plurality of infusion ports in a shaft of the guidewire proximal to a distal guidewire coil.

13. A method as in claim 12, wherein the guidewire shaft has an outer diameter in the range from 0.25 mm to 1.25 mm and a fluid delivery lumen in the range 0.15 mm to 1.00 mm.

14. A method as in claim 12, wherein the infusion ports have a width in the range form from 0.05 mm to 0.4 mm.

15. A method as in claim 12, wherein from 1 ports to 50 ports are axially spaced over a distal length in the range from 0.5 cm to 15 cm.

16. A method as in claim 12, further comprising infusing a substance into the lumen using a syringe which may be removably connected to the infusion hub.

17. A method as in claim 12, further comprising infusing a substance through a fitting on the syringe.

18. A method as in claim 12, wherein the agent is therapeutic.

19. A method as in claim 12, wherein the agent is diagnostic.

20. A method as in claim 12, wherein the agent is a thrombolytic agent.

21. A method as in claim 12, wherein the agent inhibits restenosis.
